SEQUENCE LISTING (2 sequences)

SEQ ID NO: 1 - DNA, 34 bp, Artificial Sequence
  Primer
tacattgttcttgaattaaaagtgcttgcagatg

SEQ ID NO: 2 - DNA, 26 bp, Artificial Sequence
  Primer
tctggccattcaatcattgttaaacg

(19) United States
(12) Patent Application Publication
(10) Pub. No.: US 2010/0093037 A1
(43) Pub. Date: Apr. 15, 2010

(54) GROWTH OF MICROORGANISMS IN MEDIA CONTAINING SOY COMPONENTS

(76) Inventors: KEVIN A. JARRELL, LINCOLN, MA (US); GABRIEL REZNIK, BROOKLINE, MA (US); MICHELLE A. PYNN, SHARON, MA (US)

Correspondence Address: CHOATE, HALL & STEWART LLP, TWO INTERNATIONAL PLACE, BOSTON, MA, 02110, US

(21) Appl. No.: 12/565,449

(22) Filed: Sep. 23, 2009

Related U.S. Application Data
(60) Provisional application No. 61/099,227, filed on Sep. 23, 2008.
(60) Provisional application No. 61/117,877, filed on Nov. 25, 2008.

(51) Int. Cl. C12P 21/06; C12N 1/20; C12P 13/04

(52) U.S. Cl. 435/70.1; 435/252.5; 435/106

(57) ABSTRACT

The present invention provides novel methods of growing of microorganisms in cell culture media comprising soy components (e.g., soy molasses) as a carbon source. The present invention further provides novel cell culture media comprising soy components (e.g., soy molasses) as a carbon source. In certain embodiments, inventive cell culture media substantially lack a carbon source other than soy molasses (e.g., the media substantially lack glucose and glycerol). In certain embodiments, inventive cell culture media comprise soy components (e.g., soy molasses) as the sole carbon source.

### CROSS REFERENCE TO RELATED APPLICATIONS

The present application is copending with, shares at least one common inventor with and claims priority to U.S. provisional patent application Ser. No. 61/099,227, filed Sep. 23, 2008, and to U.S. Provisional Application No. 61/117,877, filed Nov. 25, 2008. The entire contents of the prior applications are herein incorporated by reference.

### BACKGROUND

Microorganisms are typically grown in cell culture media that contain a carbon source. Carbon sources are often simple sugars such as glucose or galactose, which are broken down and converted to energy, cellular components, and/or metabolic products. The choice of which carbon source to use in the culturing of microorganisms is determined by a variety of factors including considerations such as the ability of the microorganism to utilize a particular carbon source, the ability of the microorganism to convert a particular carbon source into a product of interest, the type and amount of byproducts produced as a result of metabolizing the carbon source, the availability of a carbon source, the present and/or future cost a particular carbon source, etc.

In some cases, microorganisms are grown in cell culture media that contain glucose or refined glycerol as an energy source. Glucose is commercially produced by enzymatic hydrolysis of starches derived from crops such as maize, rice, wheat, potato, cassava, arrowroot, and sago. Refined glycerol is typically generated from crude glycerol through an intensive process that removes contaminants and impurities that are generally thought to be detrimental to the growth of microorganisms. Less expensive, alternative carbon sources are needed for economical commercial-scale production of compounds produced by microorganisms.

### SUMMARY OF THE INVENTION

The present invention provides improved compositions and methods for growing microorganisms (e.g., bacteria or fungi) in cell culture media using carbon sources from soy components (e.g., inexpensive soy components such as soy molasses). In certain embodiments, methods are provided wherein a microorganism is grown in a cell culture comprising soy components (e.g., soy molasses) as a carbon source. In certain embodiments, methods are provided wherein a microorganism is grown in a cell culture comprising soy components (e.g., soy molasses) as a carbon source, which cell culture further substantially lacks added glucose and/or glycerol (e.g., refined glycerol). In certain embodiments, methods are provided wherein a microorganism is grown in a cell culture comprising soy components (e.g., soy molasses) as the sole carbon source.

The present invention also provides improved culture media suitable for growth of microorganisms. In certain embodiments, a cell culture medium of the present invention comprises soy components (e.g., soy molasses) as a carbon source. In certain embodiments, a cell culture medium of the present invention comprises soy components (e.g., soy molasses) as a carbon source, which cell culture medium further substantially lacks added glucose and/or glycerol. In certain embodiments, a cell culture medium of the present invention comprises soy components (e.g., soy molasses) as the sole carbon source.

In some embodiments, a cell culture medium comprises soy molasses at a concentration of 1-10% solids. In some embodiments, a cell culture medium comprises soy molasses at a concentration of 3-6% solids. In some embodiments, a cell culture medium includes less than 0.1% glucose. In some embodiments, a culture medium is a liquid medium. In some embodiments, a culture medium is a solid medium. In some embodiments, a medium is a Bacillus subtilis cell culture medium.

In some embodiments, a medium comprises one or more of (NH₄)₂SO₄, K₂HPO₄, KH₂PO₄, Na₃-citrate dihydrate, magnesium sulfate heptahydrate, CaCl₂ dihydrate, FeSO₄ heptahydrate, and disodium EDTA dihydrate. For example, in some embodiments, a medium comprises: (NH₄)₂SO₄ at a concentration of about 2 g/L; K₂HPO₄ at a concentration of about 14 g/L; KH₂PO₄ at a concentration of about 6 g/L; Na₃-citrate dihydrate at a concentration of about 1 g/L; magnesium sulfate heptahydrate at a concentration of about 0.2 g/L; CaCl₂ dihydrate at a concentration of about 14.7 mg/L; FeSO₄ heptahydrate at a concentration of about 1.1 mg/L; and disodium EDTA dihydrate at a concentration of about 1.5 mg/L. In some embodiments, a medium further comprises MnSO₄ (e.g., at a concentration of about 10 μM).

In some embodiments, a medium comprises a nitrogen source, e.g., a nitrogen source selected from the group consisting of: total soy extract, tryptone, yeast extract, casamino acids, distiller grains, and combinations thereof. In some embodiments, a culture medium comprises yeast extract.

Any of a wide variety of microorganisms can be grown in inventive cell culture media that comprise soy components (e.g., soy molasses) as a carbon source. For example, any of a variety of bacteria may be grown according to the present invention. As non-limiting examples, bacteria of the genera Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix and Actinosynnema may be grown in accordance with compositions and/or methods of the present invention. In certain embodiments, a bacterium of the genus Bacillus grown is grown in accordance with compositions and/or methods of the present invention. In certain embodiments, a bacterium of the species Bacillus subtilis is grown in accordance with compositions and/or methods of the present invention.

Additionally or alternatively, any of a variety of fungi may be grown according to the present invention. Soy molasses contains carbohydrates which can be utilized by fungi. In addition, certain fungi have alpha-galactosidase which allows them to metabolize stachyose and raffinose present in soy molasses. In certain embodiments, a fungus grown in accordance with compositions and/or methods of the present invention is a yeast. As non-limiting examples, yeast of the genera Saccharomyces, Pichia, Aspergillus, Trichoderma, Kluyveromyces, Candida, Hansenula, Schizpsaccaromyces, Yarrowia, Chrysoporium, Rhizopus, Aspergillus and Neurospora may be grown in accordance with compositions and/or methods of the present invention. In certain embodiments, a yeast of the genus Saccharomyces grown is grown in accordance with compositions and/or methods of the present invention. In certain embodiments, a yeast of the species Saccharomyces cerevisiae is grown in accordance with compositions and/or methods of the present invention.

Microorganisms grown in a cell culture medium described herein can be used to produce any of a variety of products. In certain embodiments, a microorganism grown in an inventive cell culture medium and/or according to inventive methods produces a polypeptide, non-ribosomal peptide, acyl amino acid, and/or lipopeptide of interest (e.g., an acyl amino acid or lipopeptide which is a surfactant). As one non-limiting example, a microorganism grown in an inventive cell culture medium and/or according to inventive methods may produce surfactin. Those of ordinary skill in the art will be aware of other polypeptides, non-ribosomal peptides, and/or lipopeptides of interest, as well as microorganisms that produce them. Such art-recognized polypeptides, non-ribosomal peptides, acyl amino acids, and/or lipopeptides of interest can be grown in inventive cell culture media and/or according to methods of the present invention. In certain embodiments, such a microorganism produces the polypeptide, non-ribosomal peptide, acyl amino acid, and/or lipopeptide of interest to a level that is at least that of a microorganism grown in traditional cell culture media and/or according to traditional methods. In certain embodiments, the yield (defined as percent of carbon source converted into a product of interest) of a polypeptide, non-ribosomal peptide, acyl amino acid, and/or lipopeptide of interest produced by a microorganism grown in inventive media containing soy components is at least about 10%, 15%, 20%, 25%, 30%, 35%, 40%, 45%, 50%, 55%, 56%, 57%, 58%, 59% 60% or more.

In certain embodiments, a microorganism grown in an inventive cell culture medium and/or according to inventive methods that produces a polypeptide, non-ribosomal peptide, acyl amino acid, and/or lipopeptide of interest is a bacterium. As non-limiting examples, bacteria of the genera Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix and Actinosynnema may be grown in accordance with compositions and/or methods of the present invention to produce a polypeptide, non-ribosomal peptide, acyl amino acid, and/or lipopeptide of interest. In certain embodiments, such a bacterium is of the genus Bacillus. In certain embodiments, such a bacterium is of the species Bacillus subtilis.

In certain embodiments, an inventive cell culture medium comprises a nitrogen source. Nitrogen sources that can be used in accordance with the present invention include, but are not limited to, tryptone, total soy extract, yeast extract, casamino acids and/or distiller grains.

In another aspect, the present invention provides methods for growing cells (e.g., fungi or bacteria, e.g., Bacillus cells, e.g. Bacillus subtilis cells) in a cell culture. The methods include, for example, growing the cells in a cell culture medium comprising a carbon source which comprises soy components. In some embodiments, soy components comprise soy molasses. In some embodiments, a medium includes less than 0.1% glucose. In some embodiments, a medium lacks a carbon source other than soy molasses.

In some embodiments, cells (e.g., Bacillus cells) produce a lipopeptide or an acyl amino acid. In some embodiments, cells (e.g., Bacillus cells) comprise a recombinant polypeptide which produces the lipopeptide or acyl amino acid. In some embodiments, cells (e.g., Bacillus cells) produce a lipopeptide which comprises surfactin. In some embodiments, the yield of surfactin produced from the cell culture is at least about 1 g/L. In some embodiments, cells (e.g., Bacillus cells) produce an acyl amino acid, e.g., acyl glutamate. In some embodiments, methods include isolating a fraction of a cell culture comprising a produced lipopeptide or acyl amino acid. In some embodiments, methods include purifying a produced lipopeptide or acyl amino acid.

In some embodiments, a medium has less than 0.1% glucose, and wherein a cell culture produces a lipopeptide or acyl amino acid at a level comparable to, or greater than, a level of the lipopeptide or acyl amino acid produced in a culture having added glucose and which is otherwise identical.

In some embodiments, a medium comprises soy molasses at a final concentration of 1-10% solids (e.g., a medium comprises soy molasses at a final concentration of 3-6% solids).

In some embodiments, a medium is a liquid medium. In some embodiments, a medium is a solid medium. In some embodiments, a medium is a Bacillus subtilis cell culture medium.

In some embodiments of provided methods, a medium comprises one or more of (NH₄)₂SO₄, K₂HPO₄, KH₂PO₄, Na₃-citrate dihydrate, magnesium sulfate heptahydrate, CaCl₂ dihydrate, FeSO₄ heptahydrate, and disodium EDTA dihydrate. For example, in some embodiments, a medium comprises: (NH₄)₂SO₄ at a concentration of about 2 g/L; K₂HPO₄ at a concentration of about 14 g/L; KH₂PO₄ at a concentration of about 6 g/L; Na₃-citrate dihydrate at a concentration of about 1 g/L; magnesium sulfate heptahydrate at a concentration of about 0.2 g/L; CaCl₂ dihydrate at a concentration of about 14.7 mg/L; FeSO₄ heptahydrate at a concentration of about 1.1 mg/L; and disodium EDTA dihydrate at a concentration of about 1.5 mg/L. In some embodiments, a medium further comprises MnSO₄ (e.g., at a concentration of about 10 μM).

In some embodiments, a medium comprises a nitrogen source, e.g., a nitrogen source selected from the group consisting of: total soy extract, tryptone, yeast extract, casamino acids, distiller grains, and combinations thereof. In some embodiments, a culture medium comprises yeast extract.

The present invention also features methods of producing a lipopeptide or an acyl amino acid. Methods include, for example, providing a first cell culture by growing cells (e.g. Bacillus cells) that produce a lipopeptide or an acyl amino acid in a first cell culture medium, wherein the first medium comprises Na₂HPO₄, KH₂PO₄, NaCl, NH₄Cl, yeast extract, and glycerol; providing a second cell culture by inoculating a second cell culture medium with a portion of the first cell culture, wherein the second medium comprises (NH₄)₂SO₄, K₂HPO₄, KH₂PO₄, Na₃-citrate dihydrate, magnesium sulfate heptahydrate, CaCl₂ dihydrate, FeSO₄ heptahydrate, disodium EDTA dihydrate, and glucose; providing a third cell culture by inoculating a third cell culture medium with a portion or the second cell culture, wherein the third medium comprises (NH₄)₂SO₄, K₂HPO₄, KH₂PO₄, Na₃-citrate dihydrate, magnesium sulfate heptahydrate, CaCl₂ dihydrate, FeSO₄ heptahydrate, disodium EDTA dihydrate, soy molasses, and MnSO₄; thereby producing a lipopeptide or acyl amino acid.

In some embodiments, methods further include isolating a portion of the third cell culture which comprises the lipopeptide or acyl amino acid. In some embodiments, the first cell culture is grown for about 24 hours prior to inoculating the second culture. In some embodiments, the second cell culture is grown for about 24 hours prior to inoculating the third culture. In some embodiments, the third cell culture is grown for about 120 hours.

In some embodiments, a first cell culture medium comprises: Na₂HPO₄ at a concentration of about 6 g/L; KH₂PO₄ at 3 g/L; NaCl at a concentration of about 0.5 g/L; NH₄Cl at a concentration of about 1 g/L; yeast extract at a concentration of about 3 g/L; and about 0.5% glycerol.

In some embodiments, a second cell culture medium comprises: (NH₄)₂SO₄ at a concentration of about 2 g/L; K₂HPO₄ at a concentration of about 14 g/L; KH₂PO₄ at a concentration of about 6 g/L; Na₃-citrate dihydrate at a concentration of about 1 g/L; magnesium sulfate heptahydrate at a concentration of about 0.2 g/L; CaCl₂ dihydrate at a concentration of about 14.7 mg/L; FeSO₄ heptahydrate at a concentration of about 1.1 mg/L; disodium EDTA dihydrate at a concentration of about 1.5 mg/L; and glucose at about 40 g/L.

In some embodiments, a third cell culture medium comprises: (NH₄)₂SO₄ at a concentration of about 2 g/L; K₂HPO₄ at a concentration of about 14 g/L; KH₂PO₄ at a concentration of about 6 g/L; Na₃-citrate dihydrate at a concentration of about 1 g/L; magnesium sulfate heptahydrate at a concentration of about 0.2 g/L; CaCl₂ dihydrate at a concentration of about 14.7 mg/L; FeSO₄ heptahydrate at a concentration of about 1.1 mg/L; disodium EDTA dihydrate at a concentration of about 1.5 mg/L; soy molasses at a concentration of about 4% solids; and MnSO₄ at a concentration of about 10 μM.

The present invention also provides compositions including cells and a culture medium described herein. For example, the present invention provides a composition comprising Bacillus cells and cell culture medium, wherein the cell culture medium comprises a carbon source which comprises soy components. In some embodiments, soy components comprise soy molasses. In some embodiments, Bacillus cells produce a lipopeptide or acyl amino acid. In some embodiments, a composition comprises lipopeptide and/or acyl amino acids produced by the cells. Compositions comprising products of cells grown in medium provided herein are also provided.

The details of one or more embodiments of the invention are set forth in the description below. Other features, objects, and advantages of the invention will be apparent from the description and from the claims. All cited patents, patent applications, and references (including references to public sequence database entries) are incorporated by reference in their entireties for all purposes.

### DETAILED DESCRIPTION OF CERTAIN EMBODIMENTS

### Definitions

“Acyl amino acid”: The term “acyl amino acid” as used herein refers to an amino acid that is covalently linked to a fatty acid. In certain embodiments, acyl amino acids are produced in microorganisms expressing engineered polypeptides, e.g., engineered polypeptides comprising a peptide synthetase domain covalently linked to a fatty acid linkage domain and a thioesterase domain or reductase domain. In certain embodiments, acyl amino acids are produced in microorganisms expressing engineered polypeptides comprising a peptide synthetase domain covalently linked to a beta-hydroxy fatty acid linkage domain and a thioesterase domain. In certain embodiments, acyl amino acids are produced in microorganisms expressing engineered polypeptides comprising a peptide synthetase domain covalently linked to a beta-hydroxy fatty acid linkage domain and a reductase domain. In certain embodiments, an acyl amino acid produced by a method described herein comprises a surfactant such as, without limitation, an acylated glutamate, e.g., cocoyl glutamate. In certain embodiments, acyl amino acids produced by compositions and methods of the present invention comprise a beta-hydroxy fatty acid. A beta-hydroxy fatty acid may contain 3, 4, 5, 6, 7, 8, 9, 10, 11, 12, 3, 14, 15, 15, 16, 17, 18, 19, 20 or more carbon atoms. In some embodiments, a beta-hydroxy fatty acid is beta-hydroxy myristic acid, which contains 13 to 15 carbons in the fatty acid chain.

“Carbon source”: The term “carbon source” as used herein refers to a component of a cell culture medium that comprises carbon and that is utilized by a cell (e.g., a microbial cell) in culture medium for producing energy, cellular components, and/or metabolic products. Examples of carbon sources used in cell culture media include sugars, carbohydrates, organic acids, and alcohols (e.g., glucose, fructose, mannitol, starch, starch hydrolysate, cellulose hydrolysate, molasses, soy molasses, acetic acid, propionic acid, lactic acid, formic acid, malic acid, citric acid, fumaric acid, glycerol, inositol, mannitol and sorbitol).

“Crude glycerol”: The term “crude glycerol” as used herein refers to glycerol that has not been subjected to art-recognized processes that remove contaminants and/or impurities to generate “refined glycerol” (see definition of “refined glycerol”, infra). Crude glycerol is produced by a variety of natural and synthetic processes. For example, crude glycerol is produced during the process of biodiesel production. Additionally, crude glycerol is produced during the process of saponification (e.g., making soap or candles from oils or fats). Crude glycerol may be subjected to one or more processes to render it suitable and/or more advantageous for use in growing microorganisms without converting it to “refined glycerol” as the term is used herein. For example, crude glycerol may be autoclaved to sterilize it. Additionally or alternatively, crude glycerol may be subjected to a filtration step to remove solids and other large masses. Such filtration can be performed on crude glycerol itself of on a culture medium that comprises crude glycerol. Crude glycerol subjected to such processes is not “refined glycerol” as the term is used herein.

“Culture medium”: The term “culture medium” as used herein refers to any type of medium suitable for growth of a cell (e.g., a cell of a microorganism, e.g., a bacterial cell and/or a fungal cell). In some embodiments, a culture medium comprises medium in liquid form. In some embodiments, a culture medium comprises medium in solid form (e.g., solid agar).

“Lipopeptide”: The term “lipopeptide” as used herein refers to any of a variety of molecules that contain a peptide backbone covalently linked to one or more fatty acid chains. Often, lipopeptides are produced naturally by certain microorganisms. Lipopeptides can also be produced in microorganisms that are engineered to express the lipopeptides. A lipopeptide is typically produced by one or more nonribosomal peptide synthetases that build an amino acid chain without reliance on the canonical translation machinery. For example, surfactin is cyclic lipopeptide that is naturally produced by certain bacteria, including the Gram-positive endospore-forming bacteria Bacillus subtilis. Surfactin consists of a seven amino acid peptide loop, and a hydrophobic fatty acid chain (beta-hydroxy myristic acid) thirteen to fifteen carbons long. The fatty acid chain allows permits surfactin to penetrate cellular membranes. The peptide loop is composed of the amino acids glutamic acid, leucine, D-leucine, valine, aspartic acid, D-leucine and leucine. Glutamic acid and aspartic acid residues at positions 1 and 5 respectively, constitute a minor polar domain. On the opposite side, valine residue at position 4 extends down facing the fatty acid chain, making up a major hydrophobic domain. Surfactin is synthesized by the linear nonribosomal peptide synthetase, surfactin synthetase is synthesized by the three surfactin synthetase subunits SrfA-A, SrfA-B, and SrfA-C. Each of the enzymes SrfA-A and SrfA-B consist of three amino acid activating modules, while the monomodular subunit SrfA-C adds the last amino acid residue to the heptapeptide. Additionally the SrfA-C subunit includes the thioesterase domain (“TE domain”), which catalyzes the release of the product via a nucleophilic attack of the beta-hydroxy of the fatty acid on the carbonyl of the C-terminal Leu of the peptide, cyclizing the molecule via formation of an ester. Other lipopeptides and their amino acid and fatty acid compositions are known in the art, and can be produced in accordance with compositions and/or methods of the present invention. In certain embodiments, lipopeptides are produced by a method described herein in microorganisms engineered to express one or more polypeptides that participate in lipopeptide synthesis. In certain embodiments, lipopeptides produced by compositions and methods of the present invention comprise a beta-hydroxy fatty acid. A beta-hydroxy fatty acid may contain 3, 4, 5, 6, 7, 8, 9, 10, 11, 12, 3, 14, 15, 15, 16, 17, 18, 19, 20 or more carbon atoms. In some embodiments, a beta-hydroxy fatty acid is beta-hydroxy myristic acid, which contains 13 to 15 carbons in the fatty acid chain.

“Nitrogen source”: The term “nitrogen source” as used herein refers to a component of a cell culture medium that comprises nitrogen and is utilized by a cell (e.g., a microbial cell) in culture medium for growth. Examples of nitrogen sources include soy extract, tryptone, yeast extract, casamino acids, distiller grains, ammonia and ammonium salts (e.g., ammonium chloride, ammonium nitrate, ammonium phosphate, ammonium sulfate, ammonium acetate), urea, nitrate, nitrate salts, amino acids, fish meal, peptone, corn steep liquor, and the like.

“Non-ribosomal peptide”: The term “non-ribosomal peptide” as used herein refers to a peptide chain produced by one or more nonribosomal peptide synthetases. Thus, as opposed to “polypeptides” (see definition, infra), non-ribosomal peptides are not produced by a cell's ribosomal translation machinery. Polypeptides produced by such nonribosomal peptide synthetases may be linear, cyclic or branched. Numerous examples of non-ribosomal peptides that are produced by one or more nonribosomal peptide synthetases are known in the art. One non-limiting example of non-ribosomal peptides that can be produced in accordance with the present invention is surfactin. Those of ordinary skill in the art will be aware of other non-ribosomal peptides that can be produced using compositions and methods of the present invention. In certain embodiments, a non-ribosomal peptide contains one or more covalently-linked fatty acid chains and is referred to herein as a lipopeptide (see definition of “lipopeptide”, supra).

“Polypeptide”: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. For example, a polypeptide may be glycosylated. A polypeptide can comprise two or more polypeptides that function as a single active unit.

“Refined glycerol”: The term “refined glycerol” as used herein refers to glycerol is produced by subjecting crude glycerol (see definition of “crude glycerol”, supra) to art-recognized processes that remove contaminants and/or impurities. Refined glycerol is typically sold as a product that is at least 99.5% pure, although it will be recognized by those of ordinary skill in the art that the purity of refined glycerol may be lower that 99.5%. Processes to produce refined glycerol depend substantially on the type of impurities present in crude glycerol. For example, when crude glycerol is generated by hydrolysis, the starting crude glycerol is likely to be nearly 85% water, and multi-stage evaporators constructed of stainless steel are typically employed for concentration. Crude glycerol produced by other processes often has high salt content, and thin-film distillation is frequently employed. A summary containing some common purification processes is provided in Ullman's Encyclopedia of Chemical Technology, Vol. A-12, pages 480-483. As is discussed more fully herein, crude glycerol can also be produced as a byproduct of both biodiesel production and saponification. In both biodiesel production and saponification, the crude glycerol byproduct is subjected to one or more processes that remove contaminants and/or impurities to generate “refined glycerol”. As is known to those of ordinary skill in the art, such processes are laborious and time-consuming. “Crude glycerol” as the term is used herein refers to unprocessed or minimally processed glycerol that contains these and other contaminants and/or impurities. Removal of these contaminants and/or impurities results in what is defined herein as “refined glycerol”.

“Soy components”: As used herein, “soy components” include any type of compositions produced by and/or derived from, soybeans (e.g., any type of composition produced from any part of a soybean). Soy components used as a carbon source for cell culture include carbohydrates. In some embodiments, soy components used as a carbon source for cell culture comprise soy molasses.

“Soy molasses”: Soy molasses, as used herein, refers to an extract of soybeans which is rich in carbohydrates. In some embodiments, soy molasses is an alcohol extract of soybeans. In some embodiments, soy molasses is produced by aqueous alcohol extraction of defatted soybean material (e.g., defatted soybeans). In some embodiments, soy molasses is produced by extracting soybean material with an aqueous alcohol, such as aqueous ethanol, aqueous isopropanol or aqueous methanol, and by removing alcohol from the extract. In some embodiments, soy molasses contains 10%, 20%, 30%, 40%, 50%, 60%, or 70% total soluble solids. In some embodiments, soy molasses used in a composition or method described herein is sterilized (e.g., by autoclaving).

“Substantially lacks”: The term “substantially lacks” as used herein refers to the qualitative condition of exhibiting total or near-total absence of a particular component. One of ordinary skill in the biological arts will understand that biological and chemical compositions are rarely, if ever, 100% pure. Conversely, one of ordinary skill in the biological arts will understand that biological and chemical compositions are rarely, if ever, 100% free if a particular component. The term “substantially lacks” is therefore used herein to capture the concept that a biological and chemical composition may comprise a small, inconsequential amount of one or more impurities. To give but one particular example, when it is said that a cell culture medium “substantially lacks” a given component, it is meant to indicate that although a minute amount of that component may be present (for example, as a result of being an impurity and/or a breakdown product of one or more components of the cell culture medium, or as a result of being a minor component of a pre-seed culture which is inoculated into a seed or production culture), that component is nevertheless an inconsequential part of the cell culture medium and does not alter the basic properties of that cell culture medium. In certain embodiments, the term “substantially lacks”, as applied to a given component of a cell culture medium, refers to condition wherein the cell culture medium comprises less that 5%, 4%, 3%, 2%, 1%, 0.9%, 0.8%, 0.7%, 0.6%, 0.5%, 0.4%, 0.3%, 0.2%, 0.1% or less of that component. In certain embodiments, the term “substantially lacks”, as applied to a given component of a cell culture medium, refers to condition wherein the cell culture medium lacks any detectable amount of that component.

### Carbon Sources

All living organisms require a carbon or energy source for growth, production of biologically useful molecules and metabolic activity generally. Microorganisms are known to utilize a wide variety of carbon sources, many of which are simple monosaccharide and disaccharide sugars such as, for example, glucose, dextrin, lactose, sucrose, maltose, fructose, and/or mannose. Additionally or alternatively, microorganisms are known to utilize a wide variety of non-sugar carbon sources such as, for example, starch and amino acids such as glutamate.

Although each of the carbon sources listed above is used to grow microorganisms, those of ordinary skill in the art do not employ each of these carbon sources to the same extent. For example, glucose is a common carbon source for use in growing microorganisms. In addition to cost and availability, the choice of which carbon source to use in the culturing of microorganisms is determined by a variety of other factors including considerations such as the ability of the microorganism to utilize a particular carbon source, the ability of the microorganism to convert a particular carbon source into a product of interest, the type and amount of byproducts produced as a result of metabolizing the carbon source, etc. Clearly, having more options as to which carbon source to use will provide the practitioner more flexibility in choosing an appropriate and/or advantageous carbon source, depending on his or her practical, experimental, commercial and/or other needs.

The present invention encompasses the recognition that soy components, e.g., low cost soy components such as soy molasses, can be used as a carbon source, and even as a sole carbon source, for the growth of microorganisms, e.g., for the production of products such as polypeptides, non-ribosomal peptides, acyl amino acids, and/or lipopeptides. For example, the present invention demonstrates that Bacillus subtilis can be grown in cell culture medium containing soy molasses as a sole carbon source, and that production of lipopeptides by Bacillus subtilis in such medium is comparable to production in medium containing glucose as a carbon source. According to the present invention, inexpensive soy components can be converted to high value products such as surfactants (e.g., acyl amino acid and lipopeptide surfactants) in cell culture.

In certain embodiments, microorganisms are grown in inventive cell culture media that contain soy components (e.g., soy molasses) as a carbon source, which inventive cell culture media further substantially lack an additional carbon source (e.g., the media lack added glucose and glycerol). In certain embodiments, microorganisms are grown in inventive cell culture media that contain soy components (e.g., soy molasses) as the sole carbon source. In certain embodiments, microorganisms grown in inventive cell culture media that contain soy components (e.g., soy molasses) as a carbon source produce one or more compounds of interest. For example, such microorganisms may produce polypeptides, peptides, acyl amino acids, and/or lipopeptides, which can be isolated and optionally purified from the cell culture.

In certain embodiments, a cell culture medium includes soy molasses as a carbon source. Soy molasses is an industrial aqueous alcohol extract of soybeans, usually produced as a residual by-product during the production of soybean protein isolates and concentrates. Soy molasses is an inexpensive material used primarily as animal feed.

In some embodiments, soy molasses is produced by aqueous alcohol extraction of defatted soybean material, such as defatted soybean flakes, with a warm aqueous alcohol, such as aqueous ethanol, aqueous isopropanol or aqueous methanol. Thereafter the alcohol and some of the water, as is desired, are removed by methods such as evaporation, distillation, steam stripping, to obtain a substantially alcohol free soy molasses with a desired moisture content.

In some embodiments, soy molasses contains 20%, 30%, 40%, 50%, 60%, or 70% total soluble solids. The solids typically include carbohydrates, proteins and other nitrogenous substances, minerals, fats and lipoids. The major constituents of soy molasses are sugars that include oligosaccharide (stachyose and raffinose), disaccharides (sucrose) and minor amounts of monosaccharides (fructose and glucose). Minor constituents include saponins, protein, lipid, minerals (ash), isoflavones, and other organic materials. In certain embodiments, a cell culture medium includes soy molasses at a final concentration of about 1%, 2%, 3%, 4%, 5%, 6%, 7%, 8%, 9%, 10%, 11%, 12%, 13%, 14%, or 15% solids. For example, a soy molasses containing 50% solids can be added to a cell culture medium at a dilution of 1:50, 1:25, 1:16, 1:12.5, 1:10, etc.

In certain embodiments, a medium including soy components as described herein is a medium for growing microorganisms (e.g., Bacillus) in which a carbon source such as glucose is substituted with soy components (e.g., soy molasses). In certain embodiments, a medium including soy components is a modified form of a medium described by Spizizen, Proc. Nat. Acad. Sci. USA 44(10):1072-0178, 1958. In certain embodiments, a medium including soy components includes the following: (NH₄)₂SO₄, K₂HPO₄, KH₂PO₄, Na₃-citrate dehydrate, magnesium sulfate heptahydrate, CaCl₂ dihydrate, FeSO₄ heptahydrate, disodium EDTA dihydrate, and soy molasses at 2%, 3%, 4%, 5%, 6%, 7%, 8%, 9%, or 10% solids. In certain embodiments, a medium including soy components includes the following: (NH₄)₂SO₄ at 2 g/L, K₂HPO₄ at 14 g/L, KH₂PO₄ at 6 g/L, Na₃-citrate dihydrate at 1 g/L, magnesium sulfate heptahydrate at 0.2 g/L, CaCl₂ dihydrate at 14.7 mg/L, FeSO₄ heptahydrate at 1.1 mg/L, disodium EDTA dihydrate at 1.5 mg/L, and soy molasses at 2%, 3%, 4%, 5%, 6%, 7%, 8%, 9%, or 10% solids. Other media formulae suitable for growing microorganisms (e.g., Bacillus) are known and may be modified to include soy components as a carbon source in accordance with the present invention.

### Production of Polypeptides, Non-Ribosomal Peptides, Acyl Amino Acids, and Lipopeptides in Microorganisms Using Soy Components as a Carbon Source

In certain embodiments, a microorganism grown in compositions of the present invention and/or according to methods of the present invention produces one or more products of interest. For example, a microorganism may produce a polypeptide, non-ribosomal peptide, acyl amino acid, and/or a lipopeptide. As one non-limiting example, a microorganism may produce the lipopeptide surfactin. Surfactin is cyclic lipopeptide that is naturally produced by certain bacteria, including the Gram-positive endospore-forming bacteria Bacillus subtilis. Surfactin is an amphiphilic molecule (having both hydrophobic and hydrophilic properties) and is thus soluble in both organic solvents and water. Surfactin exhibits exceptional surfactant properties, making it a commercially valuable molecule. Surfactin consists of a seven amino acid peptide loop, and a hydrophobic fatty acid chain (beta-hydroxy myristic acid) thirteen to fifteen carbons long. The fatty acid chain allows surfactin to penetrate cellular membranes. The peptide loop is composed of the amino acids glutamic acid, leucine, D-leucine, valine, aspartic acid, D-leucine and leucine. Glutamic acid and aspartic acid residues at positions 1 and 5 respectively, constitute a minor polar domain. On the opposite side, valine residue at position 4 extends down facing the fatty acid chain, making up a major hydrophobic domain.

Surfactin is synthesized by the linear nonribosomal peptide synthetase, surfactin synthetase, which includes three synthetase subunits SrfA-A, SrfA-B, and SrfA-C. Each of the enzymes SrfA-A and SrfA-B consist of three amino acid activating modules, while the monomodular subunit SrfA-C adds the last amino acid residue to the heptapeptide. Additionally the SrfA-C subunit includes the thioesterase domain (“TE domain”), which catalyzes the release of the product via a nucleophilic attack of the beta-hydroxy of the fatty acid on the carbonyl of the C-terminal Leu of the peptide, cyclizing the molecule via formation of an ester.

Due to its surfactant properties, surfactin also functions as an antibiotic. For example, surfactin is known to be effective as an anti-bacterial, anti-viral, anti-fungal, anti-mycoplasma and hemolytic compound. As an anti-bacterial compound, surfactin it is capable of penetrating the cell membranes of all types of bacteria, including both Gram-negative and Gram-positive bacteria, which differ in the composition of their membrane. Gram-positive bacteria have a thick peptidoglycan layer on the outside of their phospholipid bilayer. In contrast, Gram-negative bacteria have a thinner peptidoglycan layer on the outside of their phospholipid bilayer, and further contain an additional outer lipopolysaccharide membrane. Surfactin's surfactant activity permits it to create a permeable environment for the lipid bilayer and causes disruption that solubilizes the membrane of both types of bacteria. In order for surfactin to carry out minimal antibacterial effects, the minimum inhibitory concentration (MIC) is typically in the range of 12-50 μg/ml.

In addition to its antibacterial properties, surfactin also exhibits antiviral properties, and is known to disrupt enveloped viruses such as HIV and HSV. Surfactin not only disrupts the lipid envelope of viruses, but also their capsids through ion channel formations. Surfactin isoforms containing fatty acid chains with 14 or 15 carbon atoms exhibited improved viral inactivation, thought to be due to improved disruption of the viral envelope.

Certain acyl amino acids such as sodium cocoyl glutamate also have surfactant properties. Useful acyl amino acids such as acylated glutamate, and other acylated amino acids, can be produced using media and methods described herein.

Those of ordinary skill in the art will be aware of other products (e.g., polypeptides, non-ribosomal peptides, acyl amino acids, and/or a lipopeptides) that are produced by any of a variety of microorganisms and will be able to select an appropriate microorganism to produce a product (e.g., a polypeptide, non-ribosomal peptide, acyl amino acid, and/or a lipopeptide) of interest by growing such a microorganism in compositions of the present invention and/or in accordance with methods of the present invention. In certain embodiments, a microorganism is engineered to produce a product of interest. For example, in some embodiments, a microorganism is engineered to express a polypeptide(s) that participates in the synthesis of the product of interest. In some embodiments, the polypeptide is an engineered polypeptide. In some embodiments, a microorganism that produces an acyl amino acid includes an engineered polypeptide comprising a fatty acid linkage domain, a peptide synthetase domain, and a thioesterase domain. In some embodiments, a microorganism that produces an acyl amino acid includes an engineered polypeptide comprising a fatty acid linkage domain, a peptide synthetase domain, and a reductase domain. In various embodiments, one or more of the fatty acid linkage domain, the peptide synthetase domain, and the thioesterase domain are surfactin synthetase domains. Methods of producing lipopeptides and acyl amino acids using engineered polypeptides, and methods of producing microorganisms that include the polypeptides are described in WO 2008/131002 and WO 2008/131014, the entire contents of which are hereby incorporated by reference.

In certain embodiments, a microorganism used to produce a polypeptide, non-ribosomal peptide, acyl amino acid, and/or a lipopeptide of interest when grown in compositions of the present invention and/or in accordance with methods of the present invention is a bacterium. Non-limiting examples of bacteria that can be grown in accordance with the present invention include bacteria of the genera Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix and Actinosynnema. In certain embodiments, a microorganism used to produce a polypeptide, non-ribosomal peptide and/or a lipopeptide in accordance with the present invention is a bacterium of the genus Bacillus. In certain embodiments, a microorganism used to produce a polypeptide, non-ribosomal peptide, acyl amino acid, and/or a lipopeptide in accordance with the present invention is a bacterium of the species Bacillus subtilis. Those of ordinary skill in the art will be aware of other bacteria that can produce polypeptides, non-ribosomal peptides, acyl amino acids, and/or lipopeptides when grown in compositions of the present invention and/or in accordance with methods of the present invention.

In certain embodiments, a microorganism used to produce a product of interest when grown in compositions of the present invention and/or in accordance with methods of the present invention is a fungus. Non-limiting examples of fungi that can be grown in accordance with the present invention include yeast of the genera Saccharomyces, Pichia, Aspergillus, Trichoderma, Kluyveromyces, Candida, Hansenula, Schizpsaccaromyces, Yarrowia, and Chrysoporium. Those of ordinary skill in the art will be aware of other fungi that can produce products when grown in compositions of the present invention and/or in accordance with methods of the present invention. In certain embodiments, a microorganism used in accordance with the present invention is a yeast of the genus Saccharomyces. In certain embodiments, a microorganism used in accordance with the present invention is a yeast of the species Saccharomyces cerevisiae.

Saccharomyces cerevisiae is among the first cellular organisms utilized by humans and continues to serve as a model eukaryotic organism for biological research. The extensive level of biochemical characterization of Saccharomyces cerevisiae metabolism achieved to date is a result of a thorough understanding of growth and fermentation conditions as well as the ease with which this yeast organism can be genetically manipulated. These factors combine to make this yeast organism an ideal platform for bioengineering efforts.

Growth of Saccharomyces cerevisiae requires the presence of a carbon source to support metabolic functions. Dextrose (glucose) is the preferred carbon source under aerobic conditions as an overwhelming body of evidence supports the production of metabolites to high concentrations with its use (Barnett, J. A., Payne, R. W., and Yarrow, D., Yeasts: characteristics and identification, 1st Ed., Cambridge University Press, Cambridge, 1983). However, S. cerevisiae is capable of using a variety of fermentable and non-fermentable sugars as carbon sources, increasing the versatility of this organism as an industrial platform for chemical production (see for example, Grannot and Snyder, Carbon source induces growth of stationary phase yeast cells, independent of carbon source metabolism, Yeast, May; 9(5):465-79, 1993).

Methods and compositions of the present invention expand the utility of Saccharomyces cerevisiae and other microorganisms as industrial platforms for chemical production.

In certain embodiments, Saccharomyces cerevisiae is grown in a cell culture medium comprising soy components (e.g., soy molasses) as a carbon source. In certain embodiments, Saccharomyces cerevisiae is grown in a cell culture medium that comprises soy components (e.g., soy molasses) as an energy source, which cell culture medium further substantially lacks glucose or refined glycerol. In certain embodiments, Saccharomyces cerevisiae is grown in a cell culture medium that comprises soy components (e.g., soy molasses) as the sole energy source.

In certain embodiments, a composition of the present invention used to grow a microorganism that produces one or more polypeptides, non-ribosomal peptides, acyl amino acids, and/or a lipopeptides of interest comprises a complex cell culture medium. As recognized in the art, complex media typically contain at least one component whose identity or quantity is either unknown or uncontrolled. Non-limiting examples of components that may be added to complex media include yeast extract, bacto-peptone, and/or other hydrolysates. In certain embodiments, a microorganism grown in a complex medium of the present invention comprising soy components (e.g., soy molasses) as a carbon source produces a polypeptide, non-ribosomal peptide, acyl amino acid, and/or a lipopeptide of interest in an amount that is nearly the amount of the product that would be produced if the microorganism were grown under otherwise identical conditions in a traditional complex medium. For example, a polypeptide, non-ribosomal peptide, acyl amino acid, and/or a lipopeptide produced by a microorganism in accordance with the present invention may be produced in an amount that is at least 20%, 25%, 30%, 35%, 40%, 45%, 50%, 55%, 60%, 65%, 70%, 75%, 80%, 85%, 90%, 95%, 96%, 97%, 98%, 99% or more the amount of polypeptide, non-ribosomal peptide, acyl amino acid, and/or a lipopeptide that would be produced if the microorganism were grown under otherwise identical conditions in a traditional complex medium. In certain embodiments, a microorganism grown in a complex medium of the present invention comprising soy components (e.g., soy molasses) as a carbon source produces a polypeptide, non-ribosomal peptide, acyl amino acid, and/or a lipopeptide of interest in an amount that is equivalent to the amount that would be produced if the microorganism were grown under otherwise identical conditions in a traditional complex medium. In certain embodiments, a microorganism grown in a complex medium of the present invention comprising soy components (e.g., soy molasses) as a carbon source produces a polypeptide, non-ribosomal peptide, acyl amino acid, and/or a lipopeptide of interest in an amount that is greater than the amount of polypeptide, non-ribosomal peptide, acyl amino acid, and/or a lipopeptide that would be produced if the microorganism were grown under otherwise identical conditions in a complex defined medium.

In certain embodiments, a composition of the present invention used to grow a microorganism that produces one or more polypeptides, non-ribosomal peptides, acyl amino acids, and/or a lipopeptides of interest comprises a defined cell culture medium. A variety of chemically defined growth media for use in cell culture are known to those of ordinary skill in the art. Since each component of a defined medium is typically well characterized and present in known amounts, defined media do not contain complex additives such as serum or hydrolysates. Such defined media can be modified according to the teachings of the present disclosure to generate a cell culture medium that comprises soy components (e.g., soy molasses) as a carbon source. In certain embodiments, a defined medium of the present invention comprises soy components (e.g., soy molasses) as a carbon source, and further substantially lacks a second carbon source (e.g., the medium lacks glucose or glycerol). In certain embodiments, a defined medium of the present invention comprises soy components (e.g., soy molasses) as the sole carbon source.

In certain embodiments, a defined cell culture medium of the present invention comprises a limiting amount of one or more components. As one non-limiting embodiment, a cell culture medium of the present invention may comprise a limiting amount of nitrogen.

In certain embodiments, a microorganism grown in a defined or complex medium of the present invention comprising soy components (e.g., soy molasses) as a carbon source produces a polypeptide, non-ribosomal peptide, acyl amino acid, and/or a lipopeptide to a level of 0.1 g/L, 0.2 g/L, 0.3 g/L, 0.4 g/L, 0.5 g/L, 0.6 g/L, 0.7 g/L, 0.8 g/L, 0.9 g/L, 1.0 g/L, 2.0 g/L, 3.0 g/L, 4.0 g/L, 5.0 g/L, 6.0 g/L, 7.0 g/L, 8.0 g/L, 9.0 g/L, 10 g/L, 20 g/L, 30 g/L, 40 g/L, 50 g/L, 60 g/L, 70 g/L, 80 g/L, 90 g/L, 100 g/L, or more.

In certain embodiments, the amount of polypeptide, non-ribosomal peptide, acyl amino acid, and/or lipopeptide of interest produced is increased by subjecting a cell culture containing a microorganism that produces the polypeptide, non-ribosomal peptide, acyl amino acid, and/or lipopeptide to one or more methods of the present invention. In certain embodiments, the production of a polypeptide, non-ribosomal peptide, acyl amino acid, and/or lipopeptide of interest is supplementing the cell culture with a nitrogen source such as without limitation, tryptone, total soy extract, yeast extract, casamino acids and/or distiller grains. In certain embodiments, a microorganism produces a polypeptide, non-ribosomal peptide, and/or lipopeptide of interest to an increased level relative to the level of polypeptide, non-ribosomal peptide, acyl amino acid, and/or lipopeptide that would be produced by a microorganism grown under otherwise identical conditions in an otherwise identical cell culture medium that lacks the provided nitrogen source. In certain embodiments, a nitrogen source added to the cell culture increases production of the polypeptide, non-ribosomal peptide, acyl amino acid, and/or lipopeptide of interest by a relatively greater amount than amount by which the total biomass of the cell culture is increased. In such embodiments, a polypeptide, non-ribosomal peptide, acyl amino acid, and/or lipopeptide of interest produced in a cell culture to which the nitrogen source is added represents an increased fraction of the total biomass of the cell culture compared the fraction that would result if the nitrogen source were not added to the cell culture.

In certain embodiments, the yield of a polypeptide, non-ribosomal peptide, acyl amino acid, and/or a lipopeptide of interest produced by a microorganism grown in inventive media containing soy components (e.g., soy molasses) is at least about 10%, 15%, 20%, 25%, 30%, 35%, 40%, 45%, 50%, 55%, 56%, 57%, 58%, 59%, 60%, 65%, 70%, 75%, 80%, 85%, 90% or more. Yield is defined as the amount of carbon source (e.g., soy molasses) that is converted to product (e.g., a polypeptide, non-ribosomal peptide, acyl amino acid, and/or a lipopeptide). Thus, if 50% of soy molasses is converted to a polypeptide, non-ribosomal peptide, acyl amino acid, and/or a lipopeptide, the yield is 50%.

In certain embodiments, a microorganism grown in a defined medium of the present invention comprising soy components (e.g., soy molasses) as a carbon source grows to a cell density that is comparable to the cell density that would be achieved if the microorganism were grown under otherwise identical conditions in a traditional defined medium. For example, a microorganism grown in a defined medium of the present invention comprising soy components (e.g., soy molasses) as a carbon source may grow to a cell density that is at least 50%, 55%, 60%, 65%, 70%, 75%, 80%, 85%, 90%, 95%, 96%, 97%, 98%, 99% or greater than the cell density that would be achieved if the microorganism were grown under otherwise identical conditions in a traditional defined medium. In certain embodiments, a microorganism grown in a defined medium of the present invention comprising soy components (e.g., soy molasses) as a carbon source grows to a cell density that is greater than the cell density that would be achieved if the microorganism were grown under otherwise identical conditions in a traditional defined medium. For example, a microorganism grown in a defined medium of the present invention comprising soy components (e.g., soy molasses) as a carbon source may grow to a cell density that is at least 100%, 110%, 120%, 130%, 140%, 150% or greater than the cell density that would be achieved if the microorganism were grown under otherwise identical conditions in a traditional defined medium.

### Example

A genetically engineered mutant of a strain derived from the Bacillus Genetic Stock Center, named OKB105 (sfp Phe⁻), was used in the following experiments. This strain, which is a phenylalanine auxotroph, is capable of producing the seven amino acid lipopeptide surfactin (Nakano et al., J Bacteriol. 170(12):5662-8, 1988). The ability of this strain to synthesize phenylalanine was restored by transforming it with a linear piece of DNA that was PCR-amplified using as a template total genomic DNA of Bacillus subtilis 168. The PCR reaction was carried out using the following primers:

(SEQ ID NO: 1)

| | |
|---|---|
| 23848: | 5′-TACATTGTTCTTGAATTAAAAGTGCTTGCAGATG-3′ |

(SEQ ID NO: 2)

| | |
|---|---|
| 23849: | 5′-TCTGGCCATTCAATCATTGTTAAACG-3′ |

The resulting PCR product was cleaned using a PCR Purification kit (Qiagen) and used directly to transform OKB105 competent cells. The resulting transformants were selected in (SMM). A colony that was able to grow in that media was assigned the name 028836. This strain was utilized throughout the experiments described below.

An initial set of experiments was carried out to increase the amount of surfactin produced by utilizing a modified Spizizen's minimal media (MM15). Spizizen's (unmodified) minimal media (SMM) consists of ammonium sulfate 0.2%, dipotassium phosphate 1.4%, monopotassium phosphate 0.6%, sodium citrate dihydrate 0.1%, magnesium sulfate heptahydrate 0.02%, and glucose 0.5% (Spizizen, Proc. Nat. Acad. Sci. USA, 44(10):1072-8, 1958).

The protocol used for producing surfactin includes initially growing a “pre-seed” and inoculating the pre-seed into a seed culture, which is then inoculated into a “production” media. Both pre-seed and seed are grown for 24 hrs at 30° C. Production media is grown for 120 hrs at 30° C. The pre-seed and seed are used to inoculate seed, and production media at 2% vol/vol, respectively. The media composition of the pre-seed is M9YE +0.5% glycerol (M9YE: Na₂HPO₄ 6 g, KH₂PO₄ 3 g, NaCl 0.5 g, NH₄Cl 1 g, yeast extract 3 g, water to 990 ml). The media composition of the “seed” is (NH₄)₂SO₄ 2 g, K₂HPO₄ 14 g, KH₂PO₄ 6 g, Na₃-citrate dihydrate 1 g, magnesium sulfate heptahydrate 0.2 g, glucose 40 g, CaCl₂ dihydrate 14.7 mg, FeSO₄ heptahydrate 1.1 mg, disodium EDTA dihydrate 1.5 mg per liter of water. The “production” culture is obtained by inoculating 2% of “seed” into the “production” media, which is identical to the “seed” media plus 10 μM of MnSO₄. Using this protocol, surfactin was obtained at a concentration of 1.26 g/L after three days in the production media.

To investigate the performance of soy molasses as a carbon source, the above protocol was repeated using the same pre-seed and seed, but replaced glucose in the production media with soy molasses at a final concentration of 4% solids. Soy molasses was obtained from Archer Daniels Midland Company. The batch used contained 90-95% water, 5-10% solids. For this experiment, it was assumed that 10% solids were present. Soy molasses was sterilized by autoclaving. The replacement of glucose with soy molasses yielded approximately 1.16 g/L. Data reported in this Example are the average of data collected in duplicate. These results indicate that it is possible to obtain high value products from inexpensive raw materials, derived from soy, and salts.

The foregoing description is to be understood as being representative only and is not intended to be limiting. Alternative methods and materials for implementing the invention and also additional applications will be apparent to one of skill in the art, and are intended to be included within the accompanying claims.

1. A cell culture medium for growing Bacillus cells, the cell culture medium comprising a carbon source which comprises soy components.

2. The cell culture medium of claim 1, wherein the soy components comprise soy molasses.

3. The cell culture medium of claim 1, wherein the medium includes less than 0.1% glucose.

4. The cell culture medium of claim 2, wherein the medium lacks a carbon source other than soy molasses.

5. The cell culture medium of claim 2, wherein the medium comprises soy molasses at a final concentration of 1-10% solids.

6. The cell culture medium of claim 5, wherein the medium comprises soy molasses at a final concentration of 3-6% solids.

7. The cell culture medium of claim 1, wherein the medium is a liquid medium.

8. The cell culture medium of claim 1, wherein the medium is a solid medium.

9. The cell culture medium of claim 1, wherein the medium comprises (NH₄)₂SO₄, K₂HPO₄, KH₂PO₄, Na₃-citrate dihydrate, magnesium sulfate heptahydrate, CaCl₂ dihydrate, FeSO₄ heptahydrate, and disodium EDTA dihydrate.

10. The cell culture medium of claim 9, wherein the medium comprises:
(NH₄)₂SO₄ at a concentration of about 2 g/L;
K₂HPO₄ at a concentration of about 14 g/L;
KH₂PO₄ at a concentration of about 6 g/L;
Na₃-citrate dihydrate at a concentration of about 1 g/L;
magnesium sulfate heptahydrate at a concentration of about 0.2 g/L;
CaCl₂ dihydrate at a concentration of about 14.7 mg/L;
FeSO₄ heptahydrate at a concentration of about 1.1 mg/L; and
disodium EDTA dihydrate at a concentration of about 1.5 mg/L.

11. The cell culture medium of claim 9, wherein the medium further comprises MnSO₄.

12. (canceled)

13. The cell culture medium of claim 1, wherein the medium comprises a nitrogen source selected from the group consisting of: total soy extract, tryptone, yeast extract, casamino acids, distiller grains, and combinations thereof.

14. (canceled)

15. A method for growing Bacillus cells in a cell culture, the method comprising growing the cells in a cell culture medium comprising a carbon source which comprises soy components.

16. The method of claim 15, wherein the soy components comprise soy molasses.

17. The method of claim 16, wherein the medium includes less than 0.1% glucose.

18. The method of claim 16, wherein the medium lacks a carbon source other than soy molasses.

19. The method of claim 15, wherein the Bacillus cells are Bacillus subtilis cells.

20. The method of claim 15, wherein the Bacillus cells produce a lipopeptide or an acyl amino acid.

21. The method of claim 20, wherein the Bacillus cells comprise a recombinant polypeptide which produces the lipopeptide or acyl amino acid.

22. The method of claim 20, wherein the cells produce a lipopeptide which comprises surfactin.

23. The method of claim 22, wherein the yield of surfactin produced from the cell culture is at least about 1 g/L.

24. The method of claim 20, wherein the medium has less than 0.1% glucose, and wherein the cell culture produces a lipopeptide or acyl amino acid at a level comparable to, or greater than, a level of the lipopeptide or acyl amino acid produced in a culture having added glucose and which is otherwise identical to the medium comprising a carbon source which comprises soy components.

25. The method of claim 15, wherein the medium comprises soy molasses at a final concentration of 1-10% solids.

26. The method of claim 16, wherein the medium comprises soy molasses at a final concentration of 3-6% solids.

27. The method of claim 15, wherein the medium is a liquid medium.

28. The method of claim 15, wherein the medium is a solid medium.

29. (canceled)

30. The method of claim 15, wherein the medium comprises (NH₄)₂SO₄, K₂HPO₄, KH₂PO₄, Na₃-citrate dihydrate, magnesium sulfate heptahydrate, CaCl₂ dihydrate, FeSO₄ heptahydrate, and disodium EDTA dihydrate.

31. The method of claim 30, wherein the medium comprises:
(NH₄)₂SO₄ at a concentration of about 2 g/L;
K₂HPO₄ at a concentration of about 14 g/L;
KH₂PO₄ at a concentration of about 6 g/L;
Na₃-citrate dihydrate at a concentration of about 1 g/L;
magnesium sulfate heptahydrate at a concentration of about 0.2 g/L;
CaCl₂ dihydrate at a concentration of about 14.7 mg/L;
FeSO₄ heptahydrate at a concentration of about 1.1 mg/L; and
disodium EDTA dihydrate at a concentration of about 1.5 mg/L.

32. The method of claim 30, wherein the medium further comprises MnSO₄.

33. The method of claim 32, wherein the medium comprises MnSO₄ at a concentration of about 10 μM.

34. The method of claim 15, wherein the medium comprises a nitrogen source selected from the group consisting of: total soy extract, tryptone, yeast extract, casamino acids, distiller grains, and combinations thereof.

35. The method of claim 34, wherein the culture medium comprises yeast extract.

36. A method of producing a lipopeptide or an acyl amino acid, the method comprising:
providing a first cell culture by growing Bacillus cells that produce a lipopeptide or an acyl amino acid in a first cell culture medium, wherein the first medium comprises Na₂HPO₄, KH₂PO₄, NaCl, NH₄Cl, yeast extract, and glycerol;
providing a second cell culture by inoculating a second cell culture medium with a portion of the first cell culture, wherein the second medium comprises (NH₄)₂SO₄, K₂HPO₄, KH₂PO₄, Na₃-citrate dihydrate, magnesium sulfate heptahydrate, CaCl₂ dihydrate, FeSO₄ heptahydrate, disodium EDTA dihydrate, and glucose;
providing a third cell culture by inoculating a third cell culture medium with a portion or the second cell culture, wherein the third medium comprises (NH₄)₂SO₄, K₂HPO₄, KH₂PO₄, Na₃-citrate dihydrate, magnesium sulfate heptahydrate, CaCl₂ dihydrate, FeSO₄ heptahydrate, disodium EDTA dihydrate, soy molasses, and MnSO₄;
thereby producing a lipopeptide or acyl amino acid.

37. The method of claim 36, further comprising isolating a portion of the third cell culture which comprises the lipopeptide or acyl amino acid.

38. The method of claim 36, wherein the first cell culture is grown for about 24 hours prior to inoculating the second culture.

39. The method of claim 36, wherein the second cell culture is grown for about 24 hours prior to inoculating the third culture.

40. The method of claim 36, wherein the third cell culture is grown for about 120 hours.

41-43. (canceled)

44. A composition comprising Bacillus cells and cell culture medium, wherein the cell culture medium comprises a carbon source which comprises soy components.

45. The composition of claim 44, wherein the soy components comprise soy molasses.

46. The composition of claim 44, wherein the Bacillus cells produce a lipopeptide or acyl amino acid.

47. (canceled)
